(19) United States
(12) Patent Application Publication
(10) Pub. No.: US 2024/0032160 A1
(43) Pub. Date: Jan. 25, 2024

(54) CASSETTE TO PREVENT OVERHEATING OF DIELECTRIC LOADS

(71) Applicant: CONROY MEDICAL AB, Upplands Väsby (SE)

(72) Inventor: Lars EKEMAR, Södertälje (SE)

(21) Appl. No.: 18/041,410

(22) PCT Filed: Aug. 10, 2021
(86) PCT No.: PCT/SE2021/050782

§ 371 Date: Feb. 12, 2023

(30) Foreign Application Priority Data
Aug. 11, 2020 (SE) 2000137-6

(51) Int. Cl. H05B 6/50; A61M 5/44; H05B 6/62

(52) U.S. Cl. CPC H05B 6/50; A61M 5/445; H05B 6/62; H05B 2214/02

(57) ABSTRACT

When thawing/heating sensitive organic materials such as frozen blood plasma with the help of electromagnetic fields below 400 MHz, for example, hoses and other protruding parts thaw faster than the rest of the plasma, which results in coagulated plasma. This makes the blood plasma unsuitable for transfusion. By placing the protruding portions between surfaces of metallic conductive material, these portions are protected from overheating.

### TECHNICAL FIELD

The present invention relates to a cassette to prevent overheating of dielectric loads.

### BACKGROUND

The requirements for being able to thaw and heat loads of varying sizes and of varying kinds of organic material have increased. The sizes of the loads can vary from a few tens of grams to several kilo grams, kg. The nature and sensitivity to heating of the loads can also vary.

For example, living cells such as stem cells are stored in a frozen state. The viability of the cells depends on the thawing time. The shorter the thawing time, the better the viability of the cells. In healthcare applications, there is a need for rapid thawing of blood plasma and warming of red blood cells before transfusion. These loads are very sensitive to heating, and must not, under any circumstances, even be partially heated above 40° C.

Established heating techniques, such as heating with microwaves, heating with conventional heat radiation and heating with heat convection have in common that heat absorption in load is characterized by small to negligible penetration depth and heating of load inner parts takes place by means of heat transport from heated surface portions. When thawing volumes in excess of a few milliliters of living cells, the thawing time is far too long for acceptable viability to be obtained.

It is also previously known that by emitting electromagnetic radiation/field at frequencies below 900 MHz from an antenna/antennas in a cavity with electrically conductive walls, dielectric materials placed in the cavity can be heated. (E.g. Swedish Patent 9400777-0 and Swedish patent 9703033-2). It is also known that dielectric materials can be used for field leveling purposes (E.G. European patent EP02727030.5).

Examples of this are described in document SE1450703, describing a cassette containing dielectric material (deionized water) that is in physical contact with a fragile dielectric load.

Put more simply, the cassette described in SE 1450703 can be described as a box which has a plastic bag filled with deionized water at the bottom. The delicate dielectric load, such as frozen blood plasma, is placed on top of the plastic bag. And on top of the load is placed another plastic bag filled with deionized water. Then the thawing process begins. An oscillating electric field of, for example, 150 MHz is applied. The field equalized material should have a dielectric constant that is in the range of 50 to 300 and a loss factor that is in the range of 0.04 to 2.

In practice, it has been shown that the solution works well in many cases, but in some cases problems have arisen. The temperature of the deionized water has been high compared to the temperature of the frozen blood plasma. When thawing unfolded bags of frozen blood plasma, the blood plasma contained by the tubes of the bag have thawed before the rest of the contents of the bag with frozen blood plasma. It has also been shown that the thermal contact between the upper bag with deionized water and the bag with frozen blood plasma has been uneven, which as a consequence has resulted in small local batches having thawed.

Since the thawed blood plasma has significantly higher dielectric values compared to surrounding frozen blood plasma, the consequence is that, when an oscillating electric field of, for example, 150 MHz is applied, the already thawed blood plasma with associated coagulation is heated while the frozen blood plasma remains frozen. The blood plasma thereby becomes unusable for transfusion purposes. Regardless of the type of sensitive frozen dielectric loads to be thawed, the problem remains.

There is also a need to be able to more accurately measure the heat distribution in the load during thawing. This would be possible to partially do if the upper plastic bag in the field equalizer could be completely or partially removed and the surface temperature read by IR technology.

The present invention solves these needs and problems.

### OBJECTS OF THE INVENTION

An objective of embodiments of the present invention is to provide a solution which mitigates or solves the drawbacks and problems described above.

### SUMMARY OF THE INVENTION

The above and further objectives are achieved by the subject matter described herein. Further advantageous implementation forms of the invention are further defined herein

According to a first aspect of the invention the objects of the invention is achieved by

Further applications and advantages of embodiments of the invention will be apparent from the following detailed description.

### BRIEF DESCRIPTION OF THE DRAWINGS

A more complete understanding of embodiments of the invention will be afforded to those skilled in the art, as well as a realization of additional advantages thereof, by a consideration of the following detailed description of one or more embodiments. It should be appreciated that like reference numerals are used to identify like elements illustrated in one or more of the figures.

### DETAILED DESCRIPTION

In FIG. 3 there is a cavity (H) with an applicator (G) in which electromagnetic fields are generated within the frequency range 10 Khz-600 MHz. In the roof of the cavity there are IR sensors (not drawn) that can measure the surface temperature of a bag of organic material, for example frozen blood plasma during thawing.

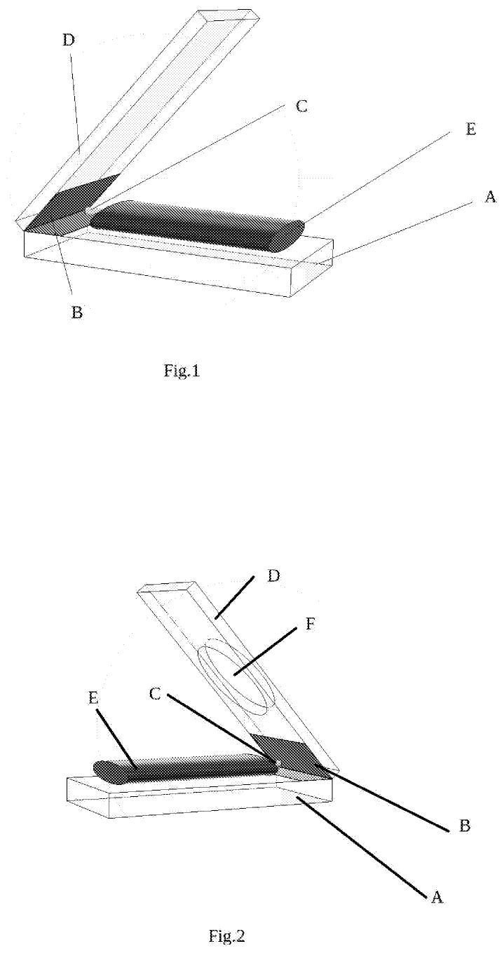

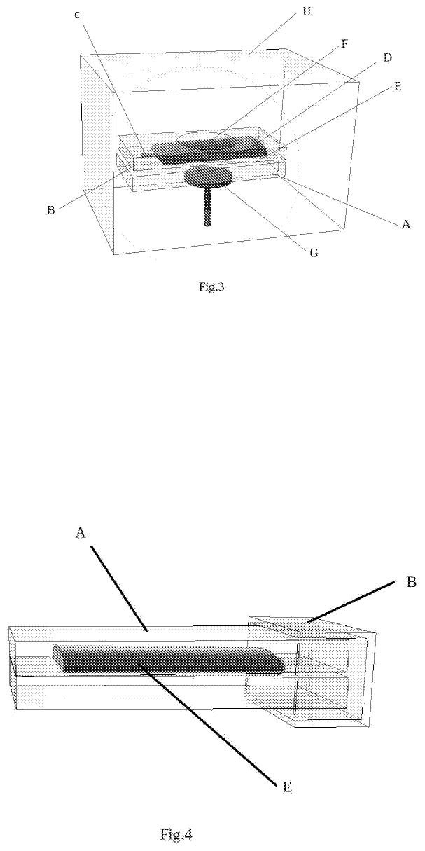

In order to be possible for temperature measurement of the bag with the organic material, there must be no field-equalized material between the load and the IR sensors. Therefore, in FIG. 2 and FIG. 3 in the upper field equalizer, a hole marked with the letter F is drawn.

Practical experience has shown that a semi-open faraday cage solves the problems mentioned in different configurations. By folding aluminum foil or other electrically conductive material over the tubes, edges and other protruding portions of the unfolded plasma bag and then placing the plasma bag on the lower container filled with deionized water and then placing a container of field leveled material over the bag of frozen organic material such as frozen blood plasma, it has been possible to thaw frozen blood plasma without coagulation occurring in tubes and other protruding portions. At the same time, the surface temperature on the upper side of the plasma bag has been able to be measured during thawing.

FIG. 1 shows a basic solution of such a field equalizer. The bag filled with organic material, for example frozen blood plasma (E), is placed in a cassette consisting of field leveling material (A and D). Tubes protruding from the bag are filled with organic material, for example frozen blood plasma is enclosed by a semi-open Faraday cage consisting of electrically conductive material. Marked as B in all figures. (You can also have a field equalizer with several Faraday cages for other/several protruding lots.)

A practical solution FIG. 1. is a cassette whose lower part consists of/is filled with a bag/container with field leveling material (For example distilled water), the surface of the lower bag is coated with a metal surface in the areas corresponding to protruding portions of organic material, for example protruding portions of a bag with frozen blood plasma.

The lid of the cassette is likewise equipped with metal surfaces, either on the upper container with field-leveled materials or directly on the lid without any field-leveled material which also corresponds to the protruding portions of a load. The metal surfaces in/on the cassette lid are in electrical conductive contact with the metal surfaces located in the lower part of the cassette. This creates a semi-open Faraday cage that protects protruding parts from overheating.

Another practical solution is shown in FIG. 2. The bag (E) with frozen or liquid organic material is placed on top of a container (A) filled with field-leveled materials. In the lid (D) there may be, but not necessarily, field-equalized dielectric material such as distilled water. There is a hole in the lid through which IR radiation can pass to be measured with the help of IR sensors in the roof of the cavity.

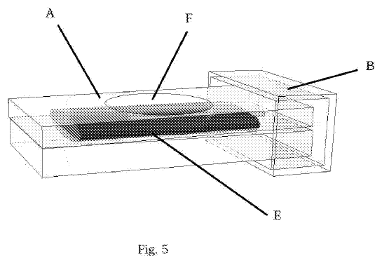

In FIG. 4 a further practical solution is described. The field equalizer (A) is partially enclosed with electrically conductive material (B) where the load (E), in this case frozen blood plasma, has a protruding portion.

This solution has the advantage that there is a softer heat distribution in the load in the area enclosed with electrically conductive material.

The weakness with that solution is that it is not possible to measure the heat distribution in the load with the help of IR sensors.

By removing, in whole or in part, the field-equalized material that exists between the organic load, in this case frozen blood plasma, and any IR sensors, it is possible to continuously measure the temperature of the load.

In FIG. 5 a practical solution is described. The load E is surrounded by field-leveling material A.

The field-leveled material A is at one end partly surrounded by electrically conductive material B. The field-leveled material is partially removed in the hole F.

Thereby it is possible to continuously measure the superficial heat distribution in the load.

Finally, it should be understood that the invention is not limited to the embodiments described above, but also relates to and incorporates all embodiments within the scope of the appended independent claims.

1. A cassette for thawing and heating of sensitive dielectric materials, which comprises an upper and lower container filled with a field equalizing material whose dielectric constant at applied frequency is less than 75 and whose loss factor is less than 250 characterized by protruding portions of the dielectric the load is partially enclosed in electrically conductive material.

2. The cassette according to claim 1, consisting of a lid and an underside, wherein parts of the lid as well as the underside consist wholly or partly of electrically conductive material.

3. The cassette according to claim 2, wherein the electrically conductive material of the lid and the bottom are in electrical contact during the thawing/heating process.

4. The cassette according to claim 1, wherein only the lower part of the cassette comprises a field-equalizing dielectric material.

5. The cassette according to claim 1, wherein a bag/container with frozen or liquid organic material is placed on top of a container filled with field equalizing material, and that in the lid, which is placed on top of the bag/container with frozen or liquid organic material, in the lid in question, there are completely or partially field-equalizing materials, and that in the lid there is a hole through which IR radiation passes to be measured.

6. A cassette for thawing and heating sensitive dielectric materials, which consists of an upper and lower container filled with a field equalizing material whose dielectric constant at applied frequency is less than 75 and whose loss factor is less than 250, wherein both containers are partially enclosed with electrically conductive material in the area/areas where the load has a protruding portion.

7. The cassette according to claim 1, consisting of a lid and an underside, wherein the field-equalized material present between the load and the IR detectors is completely or partially removed.

8. The cassette according to claim 6, consisting of a lid and an underside, wherein the field-equalized material present between the load and the IR detectors is completely or partially removed.

9. The cassette according to claim 1, wherein the field equalizing material is deionized water.

10. The cassette according to claim 6, wherein the field equalizing material is deionized water.
